(19) United States
(12) Patent Application Publication
(10) Pub. No.: US 2004/0162614 A1
(43) Pub. Date: Aug. 19, 2004

(54) Flexible auditory ossicles prosthesis

(76) Inventors: Uwe Steinhardt, Hirrlingen (DE); Karl-Bernd Huettenbrink, Dresden (DE)

Correspondence Address: STRIKER, STRIKER & STENBY, 103 East Neck Road, Huntington, NY, 11743, US

(21) Appl. No.: 10/760,017

(22) Filed: Jan. 16, 2004

(30) Foreign Application Priority Data
Jan. 17, 2003 (DE) 203 00 723.9

(51) Int. Cl. A61F 2/18

(52) U.S. Cl. 623/10

(57) ABSTRACT

An auditory ossicles prostheses has a head plate adapted to be placed on an ear drum, a base part formed so as to receive a rising bracket or to be placed in an oval window of a rising bracket foot plate, and an element for movably connecting said head part with said base part, the element for movably connecting the head part with the base part being formed as a flexible wire.

### BACKGROUND OF THE INVENTION

The present invention relates to flexible auditory ossicles prostheses.

Auditory ossicles prostheses are used, in the cases of completely or partially failing or damaged auditory ossicles of a human middle ear, to transmit the sound from the ear drum which separates the outer ear from the middle ear, to an oval window which separates the middle ear from the inner ear. In a partial prostheses the rising bracket of the auditory ossicles which is placed on the oval window is also utilized, so that the prosthesis at its end is provided with a base part which receives the rising bracket, while on the other hand a head plate is provided with which it is placed on the ear drum. In a total prosthesis it replaces completely the whole auditory ossicles. The base plate of the prosthesis is placed directly on the oval window or on the rising bracket foot plate, while the head plate of the prosthesis is placed on the ear drum.

Since anatomical characteristics of the ear, such as for example the position and shape of the rising bracket, the ear drum and the oval window, vary, and also since it can happen that after an operation with the introduction of the auditory ossicles prosthesis other position changes can occur due to the healing process by pulling forces in the tissue structures, it is advantageous to provide a certain flexibility for the auditory ossicles prosthesis.

### SUMMARY OF THE INVENTION

Accordingly, it is an object of the present invention to provide an auditory ossicles prosthesis which has a certain flexibility in a reliable, simple and wear-resistant manner.

In keeping with these objects and with others which will become apparent hereinafter, one feature of the present invention resides, briefly stated, in an auditory ossicles prostheses, comprising a head plate adapted to be placed on an ear drum, a base part formed so as to receive a rising bracket or to be placed in an oval window of a rising bracket foot plate; and means for movably connecting said head part with said base part, said means for movably connecting said head part with said base part being formed as a flexible wire.

In the inventive auditory ossicles prosthesis the head plate which is placed on the ear drum and the base part, which either in a partial prosthesis receives the rising bracket or in a total prosthesis is placed in an oval window on the rising bracket foot plate, are connected with one another by a flexible wire.

In accordance with a preferable embodiment of the present invention, the wire which connects these parts can be composed of nitinol or a spring steel.

Depending on the desired degree of flexibility and stability, the wire can have different diameters. When the diameter is selected to be in the range of 0.05 mm-0.4 mm, the prosthesis can be produced in an especially convenient manner.

Depending on the design of the head plate and the base plate, the wire which connects them can be mounted at different points of the head plate and the base plate. In the majority of the cases, the mounting provided in the center is advantageous.

With a partial prosthesis, the base part must receive the rising bracket. In this case, it can be formed with a shape of a bell and can have a springy clip, with which it can be connected in a reliable manner to the rising bracket.

A materials of the auditory ossicles prosthesis, for preventing rejection reactions, must be composed of a biocompatible material. For example, such materials can be titanium, titanium alloy, nitinol, or steel.

The novel features which are considered as characteristic for the present invention are set forth in particular in the appended claims the invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a perspective view 1 of an auditory ossicles prosthesis in accordance with one embodiment of the present invention; and

FIG. 2 is a perspective view of an auditory ossicles prosthesis in accordance with another embodiment of the present invention.

### DESCRIPTION OF THE PREFERRED EMBODIMENTS

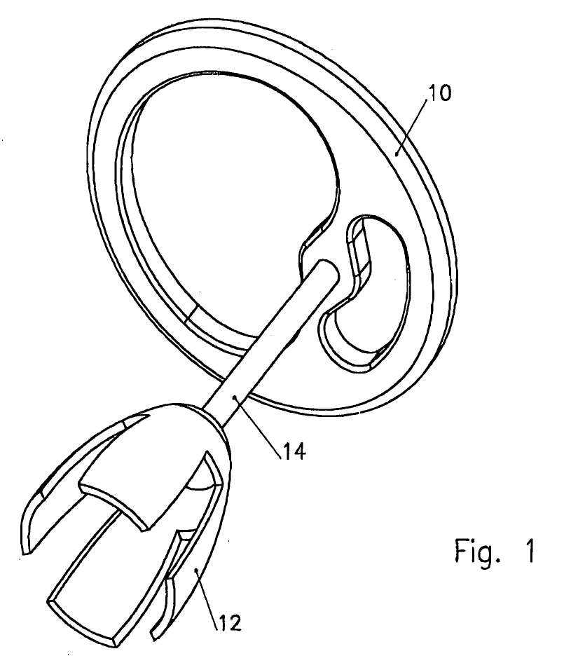

An auditory ossicles partial prosthesis in accordance with the present invention is shown on a perspective view of FIG. 1. It has one end provided for placing on an ear drum and having a head plate 10. The head plate 10 can have different shapes and must be not formed as a complete, full-surface plate.

The partial ossicles prosthesis has another end provided with a base part 12. The base part 12 must be formed so that it receives a rising bracket of a partial prosthesis. In the shown embodiment it has the shape of a bell with interrupted walls.

The head plate 10 and the base part 12 are connected with one another by a connecting element. In accordance with the present element, the connecting element is formed as a flexible wire 14. The flexible wire 14 in the shown embodiment is connected to the head plate 10 in a point which is offset from its center.

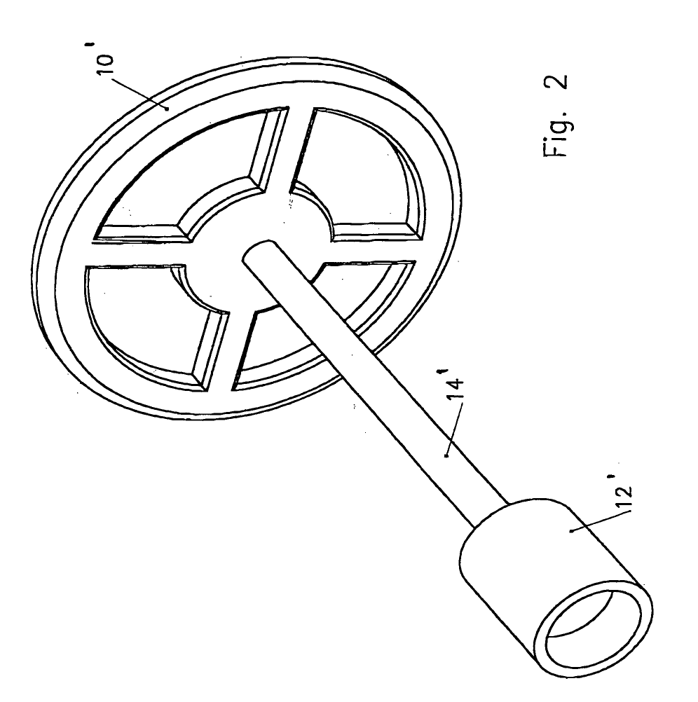

A total auditory ossicles prosthesis in accordance with the present invention is shown in a perspective view of FIG. 2. It has one end formed for placement on an ear drum and provided with a head plate 10′. The head plate 10′ in this embodiment has the shape of a cross provided with axes or legs.

In the total auditory ossicles prosthesis shown in FIG. 2 the base part 12′ is placed on an oval window directly or through a not shown rising bracket foot plate. The base part 12′ is formed as a plunger.

The head part 10′ and the base part 12′ are connected with one another by a connecting element which is formed as a flexible wire 14′. The flexible wire 14′ in the shown embodiment is connected to the head plate 10′ in the base part 12′ in their centers.

In accordance with the present invention, the wire 14, 14′ can be composed of nitinol or spring steel. The wire 14, 14′ can have a diameter from 0.05 to 0.4 mm. The head part and the base part of the inventive auditory ossicles prosthesis are composed of a biocompatible material, for example titanium, titanium alloy, nitinol, or steel.

It will be understood that each of the elements described above, or two or more together, may also find a useful application in other types of constructions differing from the types described above.

While the invention has been illustrated and described as embodied in flexible auditory ossicles prosthesis, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of the invention.

What is claimed as new and desired to be protected by Letters Patent is set forth in the appended claims:

1. An auditory ossicles prostheses, comprising a head plate adapted to be placed on an ear drum; a base part formed so as to receive a rising bracket or to be placed in an oval window of a rising bracket foot plate; and means for movably connecting said head part with said base part, said means for movably connecting said head part with said base part being formed as a flexible wire.

2. An auditory ossicles prosthesis as defined in claim 1, wherein said wire is composed of anitinol.

3. An auditory ossicles prosthesis as defined in claim 1, wherein said wire is composed of spring steel.

4. An auditory ossicles prosthesis as defined in claim 1, wherein said wire has a diameter from 0.05 to 0.4 mm.

5. An auditory ossicles prosthesis as defined in claim 1, wherein said wire is connected to said head plate in a center of said head plate and also is connected to said base part in a center of said base part.

6. An auditory ossicles prosthesis as defined in claim 1, wherein said base part is formed for receiving the rising bracket and has a shape of a bell.

7. An auditory ossicles prosthesis as defined in claim 1, wherein said base part is formed for receiving the rising bracket and includes a springy clip.

8. An auditory ossicles prosthesis as defined in claim 1, wherein said head part and said base part are composed of a biocompatible material.

9. An auditory ossicles prosthesis as defined in claim 8, wherein said biocompatible material is titanium.

10. An auditory ossicles prosthesis as defined in claim 9, wherein said biocompatible material is titanium alloy.

11. An auditory ossicles prosthesis as defined in claim 9, wherein said biocompatible material is nitinol.

12. An auditory ossicles prosthesis as defined in claim 9, wherein said biocompatible material is steel. 13. An auditory ossicles prosthesis as defined in claim 1, wherein said wire is connected with said head part in a point which is offset from a center of said head part.
